(19) United States
(12) Patent Application Publication
(10) Pub. No.: US 2022/0098677 A1
(43) Pub. Date: Mar. 31, 2022

(54) METHOD FOR DETERMINING RCC SUBTYPES

(71) Applicants: Robert Bosch Gesellschaft für medizinische Forschung mbH, Stuttgart (DE); Eberhard Karls Universität Tuebingen Medizinische Fakultaet, Tuebingen (DE); Friedrich-Alexander-Universitaet Erlangen-Nuernberg, Erlangen (DE)

(72) Inventors: Florian Buettner, Stuttgart (DE); Elke Schaeffeler, Stuttgart (DE); Matthias Schwab, Stuttgart (DE); Stefan Winter, Backnang (DE); Jens Bedke, Tuebingen (DE); Arnulf Stenzl, Tuebingen (DE); Arndt Hartmann, Erlangen (DE)

(73) Assignees: Robert Bosch Gesellschaft für medizinische Forschung mbH, Stuttgart (DE); Eberhard Karls Universität Tuebingen Medizinische Fakultaet, Tuebingen (DE); Friedrich-Alexander-Universitaet Erlangen-Nuernberg, Erlangen (DE)

(21) Appl. No.: 17/496,641

(22) Filed: Oct. 7, 2021

Related U.S. Application Data
(63) Continuation of application No. PCT/EP2020/056398, filed on Mar. 10, 2020.

(30) Foreign Application Priority Data
Apr. 12, 2019 (EP) 19169035.3

(51) Int. Cl. C12Q 1/6886

(52) U.S. Cl. CPC C12Q 1/6886; C12Q 2600/158; C12Q 2600/118; C12Q 2600/112

(57) ABSTRACT

The present invention relates to a method for determining in a subject's biological sample the relative proportions of papillary renal cell carcinoma (pRCC), clear cell renal cell carcinoma (ccRCC), and chromophobe renal cell carcinoma (chRCC), an array comprising capture molecules capable of specifically binding to RCC signature genes or coding sequences thereof or products encoded thereby, and the use of RCC signature genes for classifying a subject into a renal cell carcinoma (RCC) risk group and/or for determining in a subject's biological sample the relative proportions of pRCC, ccRCC, and chRCC.

### CROSS-REFERENCES TO RELATED APPLICATIONS

This application is a continuation of co-pending International Patent Application PCT/EP2020/056398 filed on 10 Mar. 2020 and designating the United States, which was published under PCT Article 21(2) in English, and claims priority of European Patent Application EP 19169035.3 filed on 12 Apr. 2019. All of these applications are incorporated herein by reference in their entirety.

### FIELD OF THE INVENTION

The present invention relates to a method for determining in a subject's biological sample the relative proportions of papillary renal cell carcinoma (pRCC), clear cell renal cell carcinoma (ccRCC), and chromophobe renal cell carcinoma (chRCC), an array comprising capture molecules capable of specifically binding to RCC signature genes or coding sequences thereof or products encoded thereby, and to the use of RCC signature genes for classifying a subject into a renal cell carcinoma (RCC) risk group and/or for determining in a subject's biological sample the relative proportions of pRCC, ccRCC, and chRCC.

### BACKGROUND OF THE INVENTION

Renal cell carcinoma (RCC) comprises several histologically defined tumors that differ in biology, clinical course and response to treatment. The major subtypes are clear cell RCC (ccRCC), papillary RCC (pRCC), and chromophobe RCC (chRCC), which account for 65-70%, 15-20%, and 5-7% of all RCCs, respectively (Inamura, Translocation Renal Cell Carcinoma: An Update on Clinicopathological and Molecular Features, Int. J. Mol. Sci. 9(9), p. 1-11 (2017)). In general, ccRCC has poor and chRCC has favorable prognosis. pRCC represents a heterogeneous group of RCC with intermediate prognosis compared to ccRCC and chRCC that has been subdivided in type 1 and type 2, a subset of tumors with mixed histology, and a small fraction of CpG island methylator phenotype (CIMP)-associated tumors (C. J. Ricketts et al., The Cancer Genome Atlas Comprehensive Molecular Characterization of Renal Cell Carcinoma, Cell Reports 23(1), p. 313-326 (2018)). Type 1 pRCC is associated with better prognosis than type 2 pRCC. CIMP tumors are characterized by poor survival.

Considering its significant prognostic as well as therapeutic implications, the correct determination of the subtype is of utmost importance. In clinical medicine, surgical specimen from RCC tumors are manually examined and classified by pathologists through histological and immunohistochemical analyses.

Pathological re-evaluation and bioinformatics analyses of molecular data have recently pointed to the shortcomings of pathological assessments of RCCs (Büttner et al., Survival Prediction of Clear Cell Renal Cell Carcinoma Based on Gene Expression Similarity to the Proximal Tubule of the Nephron, Eur. Urol. 68(6), p. 1016-1020 (2015); Chen et al., Multilevel Genomics-Based Taxonomy of Renal Cell Carcinoma, Cell Reports 14(10), p. 2476-2489 (2016); Schaeffeler et al., Metabolic and Lipidomic Reprogramming in Renal Cell Carcinoma Subtypes Reflects Regions of Tumor Origin, Eur. Urol. Focus (2018); C. J. Ricketts et al., loc. cit.). Manual classification is subjective and therefore bears potential for mislabeling or inconsistencies, especially in histologically ambiguous cases.

Rini et al., A 16-Gene Assay to Predict Recurrence After Surgery in Localised Renal Cell Carcinoma: Development and Validation Studies, Lancet Oncol. 16(6), p. 676-685 (2015), describe a prognostic multigene signature to improve prediction of recurrence risk in clear cell renal cell carcinoma. However, this method does not allow for an RCC subtype classification.

WO 2015/131095 discloses a method for distinguishing clear cell type A (ccA) renal cell carcinoma from clear cell type B (ccB) renal cell carcinoma in a subject. However, this method requires a statistically validated reference. Furthermore, it also does not allow for an RCC subtype classification beyond subtypes of ccRCC.

Wang et al., Identification and Validation of a 44-Gene Expression Signature for the Classification of Renal Cell Carcinomas, J. Exp. Clin. Cancer Res. 36:176, p. 1-11 (2017), disclose a 44-gene expression signature derived from microarray analysis which was associated with the histological differentiation of renal tumors and is proposed for tumor subtype classification. However, such gene expression signature has so far not proved successful in practice. Furthermore, the known method does not allow a direct subtype classification but only a clustering, i.e. individual biological samples cannot be classified.

Hence, there is a need for an objective subtype classification for RCC.

The present invention satisfies these and other needs.

### SUMMARY OF THE INVENTION

The present invention provides a method for determining in a subject's biological sample the relative proportions of papillary renal cell carcinoma (pRCC), clear cell renal cell carcinoma (ccRCC), and chromophobe renal cell carcinoma (chRCC), the method comprising:
(a) Providing a biological sample from a subject suspected of being affected by RCC,
(b) Assaying said biological sample to determine expression level values of
   at least one of the signature genes listed in Table 1,
   at least one of the signature genes listed in Table 2, and
   at least one of the signature genes listed in Table 3,
(c) Subjecting the obtained expression level values to a signal separation method, thereby determining relative proportions of pRCC, ccRCC, and chRCC in said biological sample.

The inventors have developed an objective and reference-free RCC sub-type classification system based on gene expression data by which the disadvantages of the methods known in the art can be reduced or even avoided. The present invention can also be used to separate tumors that can be unambiguously assigned to the three major histological subtypes from those combining features from different subtypes. The method according to the invention also allows a clear statement about the probability of survival of the affected patients that is more accurate and less prone to errors than a common pathological evaluation.

The present invention is superior to currently performed manual histopathological classification because (1) it provides a precise and objective molecular-based procedure to classify RCC, (2) it quantifies the proportions of the major subtypes in histologically ambiguous RCC, (3) the predicted proportional subtype composition is directly associated to a prognostic estimate, and (4) it is the first molecular-based prognostic system that is applicable to ccRCC, pRCC, and chRCC.

The term “subject” as used herein refers to a member of any invertebrate or vertebrate species. Accordingly, the term “subject” is intended to encompass any member of the Kingdom Animalia including, but not limited to the phylum Chordata (i.e., members of classes Osteichythyes (bony fish), Amphibia (amphibians), Reptilia (reptiles), Ayes (birds), and Mammalia (mammals)), and all orders and families encompassed therein. In an embodiment, the subject is a human.

A “biological sample” as used herein refers to biological material originating from the subject and comprises nucleic acids, and/or proteins, and/or peptides and/or polypeptides and/or fragments thereof. In an embodiment of the invention the biological sample comprises cellular material, cells or tissues. Preferably, the biological material comprises cells suspected of including renal carcinoma cell(s) or cells being renal carcinoma cell(s). In the clinical routine the biological sample may be a biopsy sample taken from potentially tumorous or RCC tissue, blood plasma, urine etc.

The terms “nucleic acid molecule” and “nucleic acid” refer to deoxyribonucleotides, ribonucleotides, and polymers thereof, in single-stranded or double-stranded form. As used herein, the terms “peptide” and “polypeptide” refer to polymers of at least two amino acids linked by peptide bonds. Typically, “peptides” are shorter than “polypeptides” and the latter are typically shorter than proteins, but unless the context specifically requires, these terms are used interchangeably herein.

As used herein the term “gene” refers to a hereditary unit including a sequence of DNA that occupies a specific location on a chromosome and that contains the genetic instruction for a particular characteristic or trait in an organism. Similarly, the phrase “gene product” refers to biological molecules that are the transcription and/or translation products of genes. Exemplary gene products include, but are not limited to mRNAs and polypeptides that result from translation of mRNAs.

A “signature gene” as used herein refers to a gene listed in any of Table 1, 2, and 3 and being specifically expressed and indicative for pRCC (Table 1), ccRCC (Table 2), and chRCC (Table 3), respectively. The signature genes as referred to herein make up a so-called gene signature with a unique pattern of gene expression which is characteristic in cells of ccRCC, pRCC, and chRCC.

The signature genes can be clearly identified in Tables 1, 2, and 3 by means of their GeneID, i.e. the first column of the respective table. GeneID is a unique identifier that is assigned to a gene record in the meta search engine or database ‘Entrez Gene’ operated by the National Center for Biotechnology Information (NCBI). Synonyms for ‘GeneID’ are Gene Identifies (NCBI), NCBI gene ID, Entrez gene ID, NCBI geneid, or Gene identifier (Entrez). The ‘Symbol’ column lists the HUGO Gene symbols of the genes. The columns headlined ‘ccRCC’, ‘chRCC’, and ‘pRCC’ list the medians of relative expression values of the respective signature genes in the indicated RCC subtypes. The expression values are (non-log-transformed) processed signal intensities originally measured with the Affymetrix HTA2.0 array.

As used herein “at least one” signature gene refers to the minimum of one signature gene of each Table or group that needs to be analyzed. In embodiments of the invention 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or all 58 signature genes of each Table or group are analyzed in respect of their expression levels. Further, in embodiments of the invention the numbers of signatures genes can be the same or different from each of the Tables or groups, i.e. x genes out of Table 1, y genes out of Table 2, and z genes out of Table 3 can be analyzed, while x, y, and z stand for the same or different integers.

While, according to the findings of the inventors, the analysis of one signature gene per Table is sufficient for a determination of the relative proportions of pRCC, ccRCC, and chRCC in the biological sample, the accuracy of determination and the reliability of the method is increasingly enhanced by the inclusion of more than one signature gene up to all 58 signature genes of each Table or group, respectively.

As is known to one of ordinary skill in the art, gene expression levels can be assayed at the level of RNA and/or at the level of protein. As such, in some embodiments RNA is extracted from the biological sample and analyzed by techniques that include, but are not limited to, PCR analysis (in some embodiments, quantitative reverse transcription PCR), nucleotide sequencing and/or array analysis. Alternatively or in addition, gene expression levels can be assayed by determining the levels at which proteins or polypeptides are present in the biological sample. This can also be done using arrays, and exemplary methods for producing peptide and/or polypeptide arrays attached to a suitable carrier are well known to the skilled person. In each case, one of ordinary skill in the art would be aware of techniques that can be employed to determine the expression level of a gene in the biological sample.

A “signal separation method” as used herein refers to a process for the analysis of mixtures of signals with the objective to recover the original component signals from the mixture. In particular, it is referred to a method for determining the relative proportions of ccRCC, pRCC, and chRCC in said biological sample. It includes, but is not limited to methods like blind signal separation (BSS) such as deconvolution, principal component analysis (PCA), independent component analysis (ICA), machine learning (supervised learning/classification/regression) and data mining (unsupervised learning/clustering); see Vandesompele et al., Computational deconvolution of transcriptomics data from mixed cell populations, Bioinformatics 34(11): 1969-1979 (2018).

The object underlying the invention is herewith completely solved.

The inventors have realized that determining the expression level values of only at least one of the signature genes listed in each of Tables 1, 2, and 3, i.e. of only at least three different genes, and subjecting the obtained expression level values to a signal separation method allows the determination of relative proportions of pRCC, ccRCC, and chRCC in the biological sample.

The method according to the invention allows an objective determination of the RCC subtype, thereby avoiding an incorrect subjective classification made by a pathologist. Another advantage of the method according to the invention over the methods in the art is that no reference is required to allow a correct subtype classification.

Knowledge of a pRCC versus ccRCC versus chRCC class assignment allows for an assessment of risk for recurrence or cancer specific death, and can be used to augment clinical information to make more accurate risk assessments. Knowledge of risk allows clinicians to tailor the post-operative evaluations, and to consider adjuvant therapy options. Particular changes to care that might arise when a subject's RCC is classified as comprising significant proportions of any of pRCC, ccRCC and chRCC might include, but not be limited to more intensive monitoring, consideration of surgical intervention, drug/radiation therapy, and/or finding an adjuvant therapy trial for the subject to reduce risk for recurrence.

In an embodiment of the method according to the invention in step (b) said biological sample is assayed to determine expression level values of at least two of the signature genes listed in Table 1, at least two of the signature genes listed in Table 2, and at least two of the signature genes listed in Table 3.

This measure has the advantage that the accuracy of the determination of the relative proportions of pRCC, ccRCC, and chRCC in said biological sample is further increased.

In another embodiment of the present invention the signal separation method is a blind signal separation method (BSS).

Blind signal separation (BSS), also known as blind source separation, refers to a method for the separation of a set of source signals from a set of mixed signals, without the aid of information (or with very little information) about the source signals or the mixing process. The inventors have realized that BSS, if used in the method of the invention, allows a high degree of signal separation and ensures the achievement of reliable results.

In a further embodiment of the method according to the invention the blind separation method is deconvolution, preferably computational deconvolution.

Deconvolution is an algorithm-based process used to reverse the effects of convolution on recorded data. Initially, deconvolution has been mainly used in the techniques of signal processing and image processing. Computational deconvolution refers to a computer-assisted deconvolution method which has been used to address specific questions of biology or bioinformatics, as e.g. described in S. S. Shen-Orr and R. Gaujoux, Computational Deconvolution: Extracting Cell Type-Specific Information from Heterogeneous Samples, Current Opinion in Immunology 25, p. 571-578 (2013); F. Avlia Cobos et al., Computational Deconvolution of Transcriptomics Data from Mixed Cell Populations, Bioinformatics 34, p. 1969-1979 (2018); A. R. Abbas et al., Deconvolution of Blood Microarray Data Identifies Cellular Activation Patterns in Systemic Lupus Erythematosus., PloS one 4, e6098 (2009); R. Gaujoux and C. Seoighe, CellMix: A Comprehensive Toolbox for Gene ExpressionDeconvolution., Bioinformatics (Oxford, England), p. 1-2 (2013). The inventors, however, realized for the very first time that the deconvolution method can be used in an advantageous manner to determine in a heterogeneous biological or RCC sample the relative proportions of the respective RCC subtypes.

In an embodiment of the present invention after step (c) the following step is carried out: Classifying the subject into a risk group on the basis of the relative proportions of at least one of ccRCC, pRCC, and chRCC in said biological sample, preferably of ccRCC in said biological sample.

The inventors have realized that by the developed inventive concept not only the relative proportions of the respective RCC subtypes can be determined in a biological or RCC sample but also a prediction of the risk for cancer-specific death of the patient. This measure implements the invention into the clinic in an advantageous maner.

In another embodiment of the present invention the risk group is selected from “low risk”, “intermediate risk”, and “high risk” according to the prognosis for the subject.

This measure allows a rapid allocation of a prognosis for the affected subject in daily hospital routines. “Low risk” refers to a high likelihood of the subject to survive for more than 5 years, “high risk” refers to a low likelihood of the subject to survive for more than 5 years, and “intermediate” refers to a medium likelihood of the subject to survive for more than 5 years, each 5 years period beginning to run at the date of the initial diagnosis on the basis of the biological sample from the subject obtained by surgery. In an embodiment of the invention in the “low risk” group the likelihood is about 87-96% or higher, preferably 91%, in the “high risk” group the likelihood is about 34-69%, preferably 48%, and in the “intermediate risk” group the likelihood is about 72-81%, preferably 76%.

In another embodiment of the invention the “low risk” group is determined by a relative ccRCC proportion in the range of about 0 to 12%, further preferably of about 0 to 5%, further preferably of about 0 to 3%, and highly preferably of about 0%.

In yet another embodiment of the invention the “intermediate risk” group is determined by a relative ccRCC proportion in the range of about ≥7.5 to ≤25%, further preferably of about ≥10 to ≤20%, and highly preferably of about ≥13 to ≤17.

In another embodiment of the invention the “intermediate risk” group is determined by a relative ccRCC proportion in the range of about ≥62.5%, further preferably of about ≥70%, %, further preferably of about ≥77.5%, further preferably of about 90%, and highly preferably of about 100%.

In still another embodiment of the invention the “high risk” group is determined by a relative ccRCC proportion in the range of about ≥16 to ≤77.5%, preferably of about ≥20 to ≤70%, further preferably of about ≥25 to ≤62.5%, and highly preferably of about 40%.

The inventors have realized that the indicated thresholds of the relative proportion of the respective ccRCC subtype allow an allocation of the subject to a risk group “low risk”, “high risk”, and/or “intermediate risk”. It is accepted that by using the rough or less-specific thresholds each mentioned for the less preferred embodiments a subject may fall into more than one risk group. However, it is clear that the more specific thresholds each mentioned for the more or further preferred embodiments allow an increasingly distinctive allocation of a subject to a specific risk group.

In another embodiment of the invention in step (b) the assaying involves the use of RNA sequencing, a PCR-based method, a microarray-based method, a hybridization-based method and/or an antibody-based method.

This measure takes advantage of such methods for assaying the biological sample which have been proven their suitability for determining expression level values of genes or gene products.

Another subject-matter of the present invention is an array comprising capture molecules capable of specifically binding to
biomolecules encoding or encoded by at least one, preferably at least two of the signature genes listed in Table 1 or segments thereof,
biomolecules encoding or encoded by at least one, preferably at least two of the signature genes listed in Table 2 or segments thereof, and
biomolecules encoding or encoded by at least one, preferably at least two of the signature genes listed in Table 3.

The “biomolecules” include, but are not limited hereto, nucleic acid molecules encoding the signature genes, proteins, peptides or polypeptides encoded by the signature genes. The “capture molecules” include, but are not limited hereto, nucleic acid molecules (e.g. hybridization probes, aptamers etc.), antibodies and fragments thereof.

The embodiments, features, characteristics, and advantages disclosed for the method according to the invention apply likewise to the array according to the invention.

The term “array” is to be understood in its broadest sense and refers to any kind of test format suitably adapted to comprise the capture molecules and to carry out a binding reaction of the signature genes or gene products or equivalents to the capture molecules. Preferably, the array is a microarray.

Another subject-matter of the present invention is the use of
at least one, preferably at least two of the signature genes listed in Table 1,
at least one, preferably at least two of the signature gene listed in Table 2, and
at least one, preferably at least two of the signature genes listed in Table 3,
   for classifying a subject into a renal cell carcinoma (RCC) risk group.

Another subject-matter of the present invention is the use of
at least one, preferably at least two of the signature genes listed in Table 1,
at least one, preferably at least two of the signature genes listed in Table 2, and
at least one, preferably at least two of the signature genes listed in Table 3,
   for determining in a subject's biological sample the relative proportions of papillary renal cell carcinoma (pRCC), clear cell renal cell carcinoma (ccRCC), and chromophobe renal cell carcinoma (chRCC), further preferably for classifying the subject into a RCC risk group.

The embodiments, features, characteristics, and advantages disclosed for the method according to the invention apply likewise to the uses according to the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention.

Certain features described in the context of various embodiments are not to be considered essential features of those embodiments, unless the embodiment is inoperative without those elements.

It is to be understood that the before-mentioned features and those to be mentioned in the following cannot only be used in the combination indicated in the respective case, but also in other combinations or in an isolated manner without departing from the scope of the invention.

The invention is now described and explained in further detail by referring to the following non-limiting examples and drawings.

### BRIEF DESCRIPTION OF THE DRAWINGS

FIG. 1: Overview of the data analysis workflow including different cohorts and RNA quantification technologies (microarray and RNA-Seq) used in the development of the present invention.

FIG. 2: Selection of candidate genes for the signature matrix using cohort C1. (A) Hierarchical clustering of cohort C1 (n=52) using Ward's method. (B) Tumor purity as determined by the ESTIMATE method varies between RCC subtypes. (C) The scatter plot shows P-values obtained from model comparison for each gene. The goal of the analysis was to identify genes for which expression variability is better explained by differences in RCC subtypes than by tumor purity. 28464 genes were stronger associated to RCC subtype than to tumor purity. (D) 11195 genes remained after four filtering steps. “TCGA”: genes covered in TCGA RNA-Seq data; “HG U133 Plus 2.0”: genes covered in this microarray; “exp. level”: genes with median expression above the global median in C1 in minimum one subtype; “purity ind.”: genes independently expressed of tumor purity. (E) Log2 fold changes of 3686 subtype-specific genes obtained by analysis of variance and subsequent post-hoc testing using Tukey's method. For each gene and subtype the minimum log fold change compared to the two respective other subtypes was calculated.

FIG. 3: Hierarchical clustering of cohort C2. Hierarchical clustering of cohort C2 (n=143) using Ward's method. Cohort C2 was a combined cohort containing RCC samples from five different studies (Table S1).

FIG. 4: Signature matrices with increasing number of genes were tested. The initial matrix included the top two genes per subtype exhibiting the highest log fold change compared to the respective other subtypes (FIG. 2E). The median gene expression per subtype based on cohort C1 was used. Matrix sizes ranged from 6, i.e. the top two genes per subtype, to 1500 genes. Each matrix was used to deconvolve the 143 transcriptomes from cohort C2. The maximum absolute difference (MAD) in PSA was computed between consecutive matrices for each sample. (A) The 0.95-quantile MAD between two consecutive matrices is shown. (B) Subsets including 50% of the samples were randomly drawn 10000 times from cohort C2 and for each tested matrix and subset the percentage of samples experiencing a MAD>5% compared to the predecessor matrix was determined. Matrix RCC58 including the top 58 genes per subtype was chosen (marked in light red).

FIG. 5: Receiver operating characteristic (ROC) analysis. Proportional subtype assignments (PSA) were compared with the pathological classification of the TCGA RCC cohort (Ricketts et al., Cell Reports, 2018) (n=819). The various pRCC subtypes annotated in this study were here subsumed under “pRCC”. For each of the three proportional subtype assignments (below also referred to as scores), the ability to distinguish the tumors of the corresponding subtype from the pathologically differently classified tumors was investigated in the TCGA RCC cohort for different cut-offs. AUC: area under the curve.

FIG. 6: Kaplan-Meier curves showing cancer-specific survival (CSS) of pathological subtypes (C.J. Ricketts et al., loc. cit.) in cohort C3 (n=803).

FIG. 7: Restricted cubic spline estimates of the relationships between subtype scores and log relative hazards, based on a Cox PH model with endpoint CSS. 5, 4, and 5 knots were used for fitting ccRCC-score, pRCC-score, and chRCC-score to log relative hazards, respectively. The log relative hazards were shifted in such a way that patients whose tumor was assigned a score value of 100 had a log relative hazard of zero, respectively.

FIG. 8: Risk prediction using the ccRCC score (ClearScore) in the TCGA RCC cohort (C3). (A) Relationship between ClearScore and log relative hazard for 828 patients from C3 based on cubic polynomial Cox proportional hazard modelling with endpoint CSS. 36 of 864 patients were disregarded due to lack of survival data or non-validity of the deconvolution approach (as determined by a permutation P-value estimation approach). (B) Estimated 1-, 2-, and 5-year cancer-specific survival rate in dependence of the ClearScore. (C) Distribution of ClearScore values in the different RCC subtypes defined by Ricketts et al., Cell Reports, 2018. For 789 of 828 tumors a histological classification was available (T1=type 1 pRCC, T2=type 2 pRCC, Unc.=unclassified pRCC, MD=metabolically divergent chRCC). (D) Prognostic prediction by ClearScore significantly improved pathological classification of 789 patients. Here, CIMP cases were not considered as a separate subtype but were assigned to their pathological subtypes as defined in Ricketts et al., Cell Reports, 2018. Chi-square statistic values depict the improvement of the model likelihood when estimated log relative hazards from FIG. 8A were added to the Cox model initially including the pathological classification (left) or vice versa (right). Chi-squared test P-values are shown in the bars.

FIG. 9: Graph illustrating the estimated relationship between ClearScore and the hazard ratio for endpoint cancer-specific death, using a ClearScore of 0% as reference (i.e., a ClearScore of 0%, the hazard ratio was set to 1). The hazard ratio is calculated by taking the exponential of the log relative hazards from FIG. 8A. For example, a hazard ratio of 3 means that the risk of cancer-specific death is three times higher compared to patients having a ClearScore of 0%. Risk groups were formed by categorizing the log relative hazards from FIG. 8A using conditional inference trees with endpoint cancer-specific survival. Hence, the ClearScore allows a classification of the patients into a “high risk” (top area), “low risk” (bottom area) and “intermediate risk” (middle area) group. The dashed lines indicate the pointwise standard errors. The points indicate the actual ClearScore values occurring in C3.

FIG. 10: Analysis of random signature gene subsets. 2, 5, 10, and 20 genes per subtype were randomly drawn from the 3×58 signature genes. Random drawing was repeated 10,000 times for each subset size. PSA were determined with the reduced signature gene sets. The first row shows the log-rank test P-values from univariate survival analyses in the TCGA RCC cohort (n=847) with subtype proportions, i.e. ccRCC-score, pRCC-score or chRCC-score, as predictors. Restricted cubic splines were used to model the relationship between scores and CSS. The second row shows area under the curve (AUC) values from receiver operating characteristics (ROC) analyses for each score and subset size. PSA were compared with the histological classification of the TCGA RCC cohort published by Ricketts et al., Cell Reports, 2018, (n=819). The various pRCC subtypes annotated in this study were here subsumed under “pRCC”.

### EXAMPLES

### 1. Overview

A subtype classification system based on gene expression data was developed for renal cell carcinoma (RCC). The basic idea was to model any RCC sample as a linear combination of clear cell RCC (ccRCC), chromophobe RCC (chRCC) and papillary RCC (pRCC). More than 95% of all RCC are assigned to one of these subtypes based on histological analysis and they represent both proximal and distal cell types as origin of kidney cancer evolution. Essentially, the inventors assumed a tumor not necessarily belonging to only one of these subtypes, but to carry parts of each of them. Therefore, rather than categorizing a tumor into one of the subtypes, the inventors intended to break down its composition through proportional subtype assignments (PSA).

Applying the linearity assumption (Y. Zhao and R. Simon, Gene Expression Deconvolution in Clinical Samples. Genome Med. 2(12), p. 93 (2010), the expression of each gene in the RCC sample to be analyzed can be modeled as weighted average of the expression of this gene in ccRCC, pRCC and chRCC.

The inventors realized that signal separation, in particular computational deconvolution represented the method of choice for this problem. The weights correspond to the proportional composition and are estimated by computational deconvolution. The objective of deconvolution is to find a solution to the system of linear equations: m=f×S. Here, the unknown proportions of ccRCC, pRCC and chRCC in a sample A are modeled by the vector f of coefficients. m represents the vector containing the expression levels of signature genes in A. S is a signature matrix including the expression levels of the signature genes in ccRCC, pRCC and chRCC. Signature genes are defined based on a set of ccRCC, pRCC and chRCC samples that could be uniquely assigned by pathologists or previous analyses of molecular data. The matrix equation can be solved for f using standard linear least squares regression (Abbas et al., loc. cit.). To increase stability of subtype assignments we use robust linear regression as implemented in the “rlm” function from the R-package MASS. Deconvolution is performed on linear, i.e. non-log-transformed, expression data as suggested by (Y. Zhong and Z. Liu, Gene expression deconvolution in linear space. Nat. Methods 9(1), p. 8-9 (2011)). Further, linear expression levels are centered to zero mean and scaled to unit variance preceding deconvolution. Negative coefficients in f are set to zero and percentages are calculated by dividing the three estimated coefficients by their sum.

Gene expression deconvolution has been successfully applied to characterize the cell composition of heterogeneous samples, e.g. peripheral blood that includes many different immune cell types (S.S. Shen-Orr and R. Gaujoux, loc. cit.). Here, RCC were modeled as heterogeneous tissues that are composed of varying proportions of ccRCC, chRCC and pRCC.

This study did groundwork by attempting for the first time to comprehensively detect and quantify clear as well as composed signals in RCC samples indicative of the tumor type. Methodologically, an semi-supervised approach has been developed to utilize yet unknown patterns in gene expression profiles of RCC samples for subtype classification.

The inventors' approach is able to separate RCC tumors that can be unambiguously assigned to one of the main histological subtypes from those evading a clear histological classification. Unclear tumors were described as mixed types that combine features from different subtypes. Further, PSA enabled a new definition of RCC risk groups that is significantly stronger associated with patient survival than common pathological classification. Concluding, PSA as determined by the method according to the invention simplifies classification of RCC and specifies prognosis.

### 2. Material and Methods

### Patient Cohorts

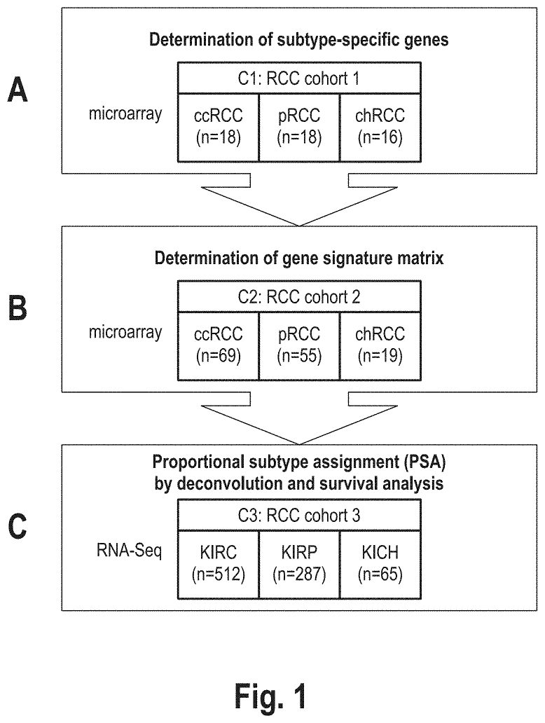

FIG. 1 shows the cohorts and their use in this work.

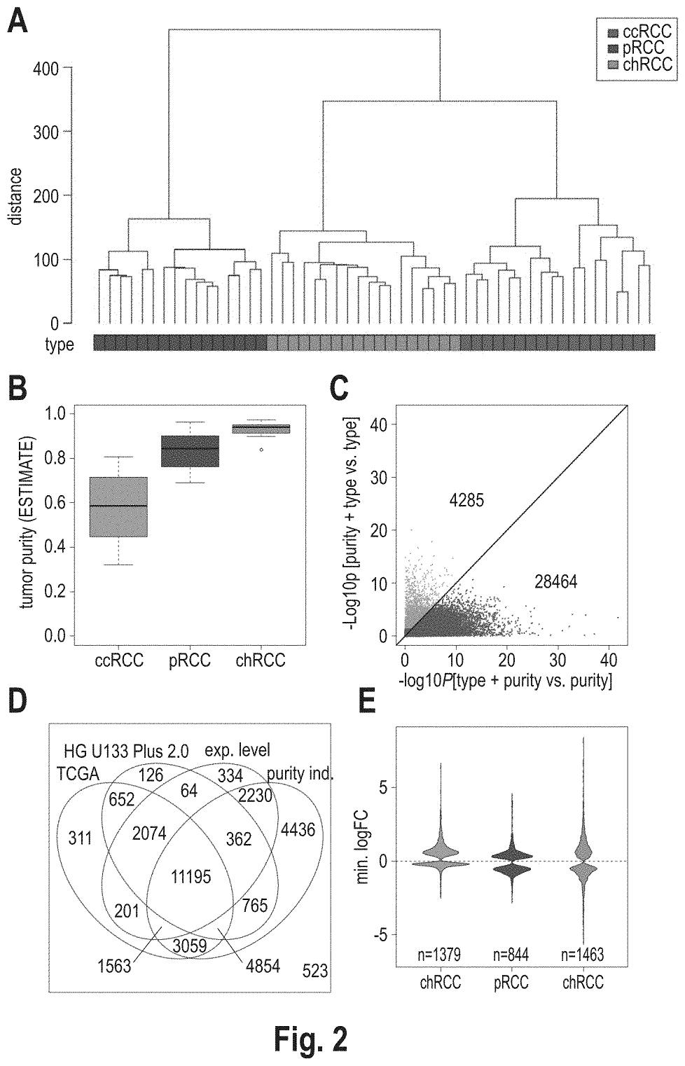

RCC cohort 1 (C1) consisted of 52 primary tumor samples with either clear cell (n=18), papillary (n=18), or chromophobe RCC histology (n=16); see FIG. 2A. All of these were collected from patients treated at the Department of Urology, University Hospital Tübingen, Germany. Use of the tissue was approved by the ethics committee of the University of Tuebingen and informed written consent was provided by each subject prior to surgical resection. Surgically resected ccRCC tissues were classified according to the seventh edition of the Union Internationale Contre Ie Cancer/American Joint Committee on Cancer system (2009). None of the patients received any kind of neoadjuvant therapy before surgery, neither immune- nor chemotherapy. Importantly, these samples have been independently evaluated by two teams of pathologists with special expertise in nephro-pathology to have maximum certainty regarding their RCC subtype. C1 was used to identify genes with RCC subtype-specific expression.

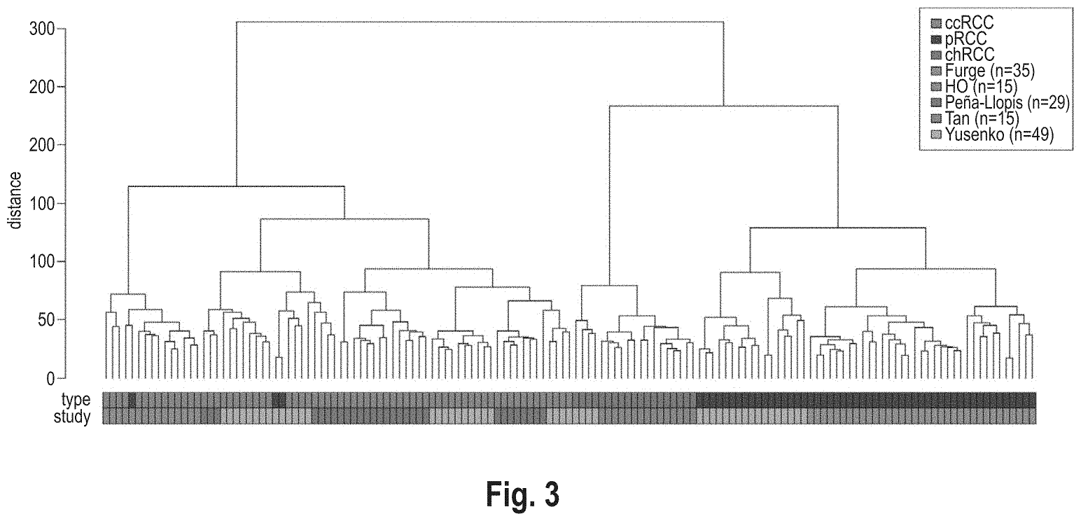

RCC cohort 2 (C2) is a combined cohort containing 143 RCC samples from five studies (K. A. Furge et al., Detection of DNA Copy Number Changes and Oncogenic Signaling Abnormalities from Gene Expression Data Reveals MYC Activation in High-Grade Papillary Renal Cell Carcinoma. Cancer Res. 67(7), p. 3171-3176 (2007); M.-H. Tan et al., Genomic Expression and Single-Nucleotide Polymorphism Profiling Discriminates Chromophobe Renal Cell Carcinoma and Oncocytoma. BMC Cancer, 10:196 (2010); S. Peña-Llopis et al., BAP1 Loss Defines a New Class of Renal Cell Carcinoma. Nat. Genet. 44(7), p. 751-759 (2012); M. V. Yusenko et al., High-resolution DNA Copy Number and Gene Expression Analyses Distinguish Chromophobe Renal Cell Carcinomas and Renal Oncocytomas. BMC Cancer 9, p. 152 (2009); T. H. Ho et al., Differential Gene Expression Profiling of Matched Primary Renal Cell Carcinoma and Metastases Reveals Upregulation of Extracellular Matrix Genes. Ann. Oncol. Off. J. Eur. Soc. Med. Oncol. 28(3), p. 604-10 (2017)); see FIG. 3. Common to these studies was the use of the Affymetrix GeneChip HG U133 Plus 2.0 for quantification of gene expression. Only samples from primary tumor tissue that were labeled ccRCC, chRCC or pRCC in the original studies were added to C2. Table S1 shows the numbers of samples per subtype obtained from each study. C2 was used to determine the signature.

TABLE S1
Cohort C2 (n = 143) includes RCC samples from
five studies providing gene expression data on Gene
expression omnibus. Expression measurements were
conducted with Affymetrix microarray HG U133 Plus 2.0.

| Study | GEO accession | ccRCC | pRCC | chRCC |
|---|---|---|---|---|
| Yusenko et al. | GSE11151 | 26 | 19 | 4 |
| Tan et al. | GSE19982 | 0 | 0 | 15 |
| Peña-Llopis et al. | GSE36895 | 29 | 0 | 0 |
| Furge et al. | GSE7023 | 0 | 35 | 0 |
| Ho et al. | GSE85258 | 14 | 1 | 0 |

Using the established signature matrix, transcriptomes from the TCGA RCC cohort (C3) were deconvolved. Clinical information and gene expression data (“FPKM-UQ″”) generated by RNA-Seq from kidney cancer cohorts KIRC, KICH and KIRP from TCGA were downloaded on Sep. 25, 2019 from https://gdc.cancer.gov/ using R-package TCGAbiolinks. XML-structured clinical information was processed using R-package XML. Disease-specific survival outcome data for the TCGA RCC cohort was obtained from (Liu et al., Cell, 2018) and was referred to as cancer-specific survival (CSS) in this work. Four patients from the KIRC cohort were repre-sented by several samples in the expression data set. The sample with highest median expression was chosen, respectively. To ensure that only tumor samples were included, remaining tumor and non-tumor samples from the TCGA RCC cohort were hierarchically clustered using Ward's method. Three cases (TCGA-BQ-5889, TCGA-CJ-5683, TCGA-DV-5573) were wrongly assigned as tumor tissues were excluded. In case of TCGA-CW-5591 tumor and non-tumor data had been confused. Patients that received prior treatment were excluded. In total the C3 cohort included tumor samples from 864 patients (KIRC: 512, KIRP: 287, KICH: 65), with survival data available for 847 patients.

### Analysis and Processing of Gene Expression Data

High quality total RNA was isolated from fresh-frozen RCC tissue from cohort C1 using the mirVana™ miRNA Isolation Kit (Life Technologies) as previously described (P. Fisel et al., DNA Methylation of the SLC16A3 Promoter Regulates Expression of the Human Lactate Transporter MCT4 in Renal Cancer with Consequences for Clinical Outcome. Clin. Cancer Res. 19(18), p. 5170-5181 (2013), S. Winter et al., Methylomes of Renal Cell Lines and Tumors or Metastases Differ Significantly with Impact on Pharmacogenes. Sci Rep. 6(1) (2018). Genome-wide transcriptome analyses were performed using the Human Transcriptome Array HTA 2.0 (Affymetrix) according to the manufacturer's protocol. Further processing of microarray data was performed as previously described (S. Winter et al., loc. cit.). Array quality control was conducted by Affymetrix Expression Console (Build 1.4.1.46). The microarrays from C1 were preprocessed together using the Robust Multiarray Average (RMA) implementation from the R-package oligo and probe sets were summarized on Entrez GeneID level using the annotation for the HTA 2.0 microarray provided by brainarray (http://brainarray.mbni.med.umich.edu, version 23).

Genome-wide transcriptome measurements performed by Affymetrix GeneChip HG U133 Plus 2.0 from 143 RCC patients included in C2 were downloaded from Gene Expression Omnibus (GEO) using R-package GEOquery (Table S1). Microarrays from C2 were normalized individually using the SCAN method from the R-package SCAN. UPC and probe sets were summarized on Entrez GeneID level using the annotation for the GeneChip HG U133 Plus 2.0 microarray provided by brainarray (http://brainarray.mbni.med.umich.edu, version 23).

Entrez GeneIDs were used as gene identifiers in this work. Probesets were summarized on Entrez GeneID level using annotations provided by brainarray (http://brainarray.mbni.med.umich.edu, version 23). Ensemble gene identifiers used in TCGA expression data were mapped to Entrez GeneIDs by means of the org.Hs.eg.db annotation package.

### Statistical Tools

All statistical analyses were performed with R-3.6.1 including additional packages beanplot 1.2, MASS_7.3-51.4, partykit_1.2-5, pROC_1.15.3, RColorBrewer_1.1-2, rms_5.1-3.1 squash_1.0.8, survival_2.41.1, and XML_3.98-1.20. GEOquery_2.46.15, oligo_1.48.0, org.Hs.eg.db_3.8.2, SCAN.UPC 2.26.0 SummarizedExperiment_1.14.1, and TCGAbiolinks_2.12.62 are part of the Bioconductor software project (http://www.bioconductor.org). All statistical tests were two-sided. Statistical significance was defined as P-value<0.05.

In hierarchical cluster analyses Euclidean distance and Ward's method have been used if not stated otherwise.

### Outcomes

Cancer-Specific Survival (CSS) was Used as an Endpoint in Survival analyses involving cohort C3. CSS time was defined as the time from initial diagnosis to death or last date of follow-up if alive. Data for patients who died from other causes than RCC disease were considered censored at the time of death.

### Gene Expression Deconvolution Using Robust Linear Regression

In this work, RCC samples were considered as mixtures of ccRCC, chRCC and pRCC. Further, it was assumed that the proportional composition of the three main subtypes is reflected in the gene expression profile of the mixed samples. According to the linearity assumption (Y. Zhao and R. Simon, loc. cit.), the expression of each gene in a mixed RCC sample can thus be modeled as weighted average of the expression of this gene in ccRCC, chRCC and pRCC. The weights correspond to the respective proportional composition that can be estimated by gene expression deconvolution.

The objective of deconvolution is to find a solution to the system of linear equations: m=S·f. Here, the unknown proportions of ccRCC, pRCC and chRCC in a sample A are modeled by the vector f of coefficients. m represents the vector containing the expression levels of signature genes in A. S is a signature matrix including the expression levels of the signature genes in ccRCC, pRCC and chRCC. Signature genes were defined based on a set of ccRCC, chRCC and pRCC samples that could be uniquely assigned by pathologists or previous analyses of molecular data. The matrix equation can be solved for [symbol] using standard linear least squares regression (A. R. Abbas et al., loc. cit.).

To increase stability of subtype assignments robust linear regression as implemented in the “rlm” function from the R-package MASS (parameter maxit was set to 200) was used in this work. Expression deconvolution was performed on linear, i.e. non-log-transformed, expression data as suggested by (Zhong et al., loc. cit.). Further, expression values were centered to zero mean and scaled to unit variance preceding deconvolution. Negative regression coefficients were set to zero and percentages were calculated by dividing the three estimates by their sum, such that c+p+h=100%, with c, p, and h representing the ccRCC, pRCC, and chRCC proportion, respectively.

An permutation P-value was calculated to assess the specificity of the signature for a certain RCC sample. Basically, the P-value computation was carried out in the same way as described in (A. M. Newman et al., Robust enumeration of cell subsets from tissue expression profiles. Nat. Methods 12(5), p. 453-457 (2015).). Briefly, Pearson's correlation coefficient R between m and S·f was compared against a derived null distribution R* [symbol] * for sample A. Expression levels in m were replaced by randomly drawn values from the complete transcriptomic data of A, denoted mᵢ* [symbol] ᵢ*. Subtype proportions fᵢ* were determined for mᵢ* by deconvolution and Pearson's correlation coefficient between mᵢ* [symbol] ᵢ* and S·fᵢ* was calculated. This process was repeated 9999 times, yielding R*, and the P-value was obtained by (|R*>R|+1)/(9999+1).

### Selection of Candidate Genes

Samples from C1 were considered as clear cases since they could unambiguously be assigned to one of the main subtypes (FIG. 2A). Transcriptome-wide RNA expression in C1 was quantified using Human Transcriptome Array 2.0 microarray technology and annotated using the brainarray annotation resulting in expression levels for 32749 genes.

Expression data from C1 were sample-wise centered to the median expression level of C1. Genes with median expression below the cohort median in each of the three subtypes were removed. Further, genes not covered in TCGA RNA expression data (https://gdc.cancer.gov/) or by the Human Genome U133 Plus 2.0 Array were excluded. It has been demonstrated using TCGA data that RCC subtypes vary in tumor purity (Yoshihara et al., Nat Commun., 2013), see also http://bioinformatics.mdanderson.org/estimate/. This pattern could be observed in C1 as well (FIG. 2B). To minimize dependence on tumor purity, genes stronger related to tumor purity, as determined by the ESTIMATE method (Yoshihara et al., Nat Commun., 2013) than to tumor type were removed. To be precise, for each gene linear regression models were fit incorporating either tumor type or tumor purity as single predictor or both of them in a multiple regression model. In the latter case models with and without interaction effects were considered. The reduction in the residual sum of squares was compared by analysis of deviance tests. Per variable, i.e. purity or type, main and main+interaction effect were tested and the lower P-value was used. A gene was kept in case its expression in cohort C1 could be better explained by tumor type than by tumor purity (FIG. 2C). 11195 genes remained after these filtering steps (FIG. 2D) and were subsequently tested for subtype-specific expression by analysis of variance. 5881 genes showed significant variation between subtypes after correction for multiple testing using Holm's method. Subsequent pairwise comparisons between subtypes by Tukey's test revealed 3686 genes to be specifically expressed either in ccRCC (1379), pRCC (844) or chRCC (1463). Median expression per subtype was determined for the candidate genes and the minimum absolute log fold change compared to the two respective other entities was calculated using the absolute values of the log fold changes (FIG. 2E). Genes were ordered by decreasing minimum absolute log fold change per subtype and expression levels were transformed to linear space

### Selection of the Signature Matrix

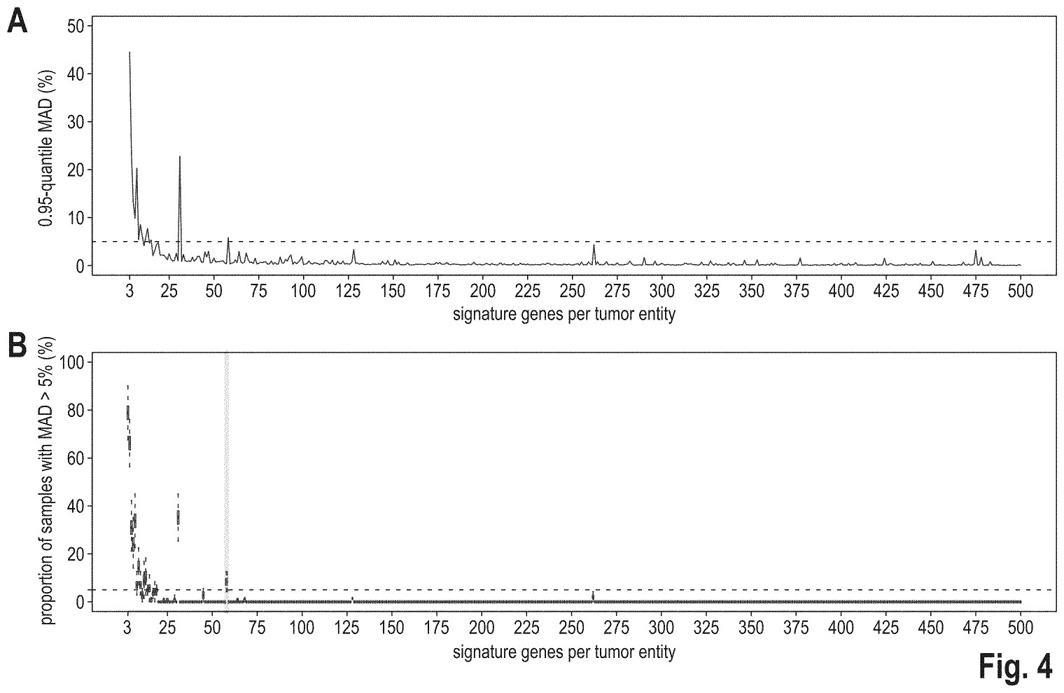

Given the set of 3686 tumor-specific genes, a sufficient subset was extracted to uncover the proportional composition of tested RCC samples. Besides distinguishing between histologically distinct ccRCC, chRCC or pRCC cases, the goal was to establish a method also being able to identify heterogeneous tumors. As in similar studies (A. R. Abbas et al., loc. cit., A. M. Newman et al., loc. cit., T. Gong et al., Optimal deconvolution of transcriptional profiling data using quadratic programming with application to complex clinical blood samples. PloS One 6(11), p. e27156 (2011), various signature matrices were created and compared (FIG. 4).

The top [symbol] n genes with the highest log fold change per subtype were combined into a signature matrix Sₙ, i.e. Sₙ included 3×n different genes. Each matrix Sₙ was used to perform a subtype prediction in cohort C2 (FIG. 3). n was iterated from 2 to 500 and for n>2 the difference in the subtype assignments between two consecutive signature matrices Sₙ and Sₙ₋₁ were calculated for each sample. The maximum absolute difference (MAD) between two assignments for a sample, i.e. max(|cₙ−cₙ₋₁|,|pₙ−pₙ₋₁|,·|hₙ−hₙ₋₁|), was used for this.

FIG. 4A shows the 0.95-quantile MAD between consecutive signature matrices. The final signature matrix was determined using a heuristic approach. Based on the assumption that more included genes allow for a more precise estimation, the largest matrix was chosen, that led to a relevant MAD in the classification (MAD>5%) for a substantial portion of C2. To strengthen the decision basis, different cohort compositions were simulated by subset sampling. In total 10,000 times 50% subsets were randomly drawn from C2. FIG. 4B shows for each matrix S_n the proportions of the sampled subsets that experienced a MAD>5% compared to the previous matrix. S_58, including 174 genes, was the largest matrix significantly modifying a substantial portion of the samples relative to the predecessor matrix (on average 8.5% per sampled subset) and therefore has been chosen as signature matrix.

### 3. Results

### Definition of Gene Signature Matrix for Deconvolution of RCC

Using the 52 RCC from cohort C1 comprising 18 ccRCC, 16 chRCC and 18 pRCC cases, each of which could be uniquely assigned by two independent teams of pathologists, candidate genes for the signature matrix were determined (FIG. 2A). To be shortlisted genes needed to be present in TCGA RNA-Seq data as well as on Affymetrix platforms HTA 2.0 and HG U133 Plus 2.0. Further, median expression had to be above the global median expression in C1 in at least one subtype. By means of TCGA RCC cohorts K. Yoshihara et al. loc. cit. have shown that tumor purity explicitly varies between RCC subtypes which could also be observed in C1 (FIG. 2B). The envisaged signature should be independent of tumor purity to be able to classify beside primary tumor tissue homogeneous tumor cells as well. Therefore, genes more related to tumor purity, as determined by the ESTIMATE method (K. Yoshihara et al. loc. cit.), than to tumor type were excluded (FIG. 2C).

11195 genes remained after filtering and were tested on differential expression between RCC subtypes by analysis of variance (FIG. 2D). Subsequent post-hoc testing using Tukey's method revealed 1379 genes to be specifically expressed in ccRCC, 1463 in chRCC, and 844 in pRCC (FIG. 2E). From this set of specifically expressed genes, signature genes have been selected by evaluating various signature gene matrices. Starting with the genes exhibiting highest minimal absolute log fold change, respectively, iteratively matrices with increasing number of genes were created and applied to deconvolve the 143 samples from cohort C2 (FIG. 3). Robust linear regression was employed for deconvolution. Matrices consisted of median expression values per RCC subtype calculated from C1. Matrix sizes ranged from 6, i.e. top two genes per subtype, to 1500 genes (FIG. 4A). Since RCC samples with defined histological composition were not available, supervised approaches could not be used to define the optimal signature. Therefore, a heuristic criterion was applied: Based on the assumption that resolution of deconvolution increases with the number of genes considered, the largest matrix achieving substantial change in subtype deconvolution compared to its predecessor matrix was chosen. If assigned proportional composition differed by more than 5% for a sample between consecutive matrices the change was considered substantial. Different cohort compositions were simulated by 50% subset sampling from C2 and the fraction of samples affected by substantial change was determined for each tested matrix. In this way, matrix RCC58 including the top 58 genes per RCC subtype was chosen as final signature (FIG. 4B).

Table 1 lists the top 58 genes for determining the pRCC subtype, Table 2 lists the top 58 genes for determining the ccRCC subtype, and Table 3 lists the top 58 genes for determining the chRCC subtype. ‘GeneID’ refers to the identifier that is assigned to a gene record in the ‘Entrez Gene’ database. The ‘Symbol’ column lists the HUGO Gene symbol of the genes. The columns headlined ‘ccRCC’, ‘chRCC’, and ‘pRCC’ list median expression values of the respective signature genes in the indicated RCC sub-types. The expression values are (non-log-transformed) processed signal intensities measured with the Affymetrix HTA2.0 array.

TABLE 1
pRCC-specific

| GeneID | Symbol | ccRCC | pRCC | chRCC |
|---|---|---|---|---|
| 164312 | LRRN4 | 39 | 815 | 40 |
| 6590 | SLPI | 68 | 1142 | 69 |
| 10568 | SLC34A2 | 120 | 1901 | 51 |
| 8842 | PROM1 | 34 | 628 | 42 |
| 5649 | RELN | 23 | 331 | 24 |
| 25928 | SOSTDC1 | 12 | 210 | 16 |
| 5284 | PIGR | 69 | 2130 | 174 |
| 27255 | CNTN6 | 14 | 157 | 16 |
| 80736 | SLC44A4 | 102 | 998 | 65 |
| 7348 | UPK1B | 23 | 184 | 23 |
| 5950 | RBP4 | 74 | 558 | 70 |
| 1365 | CLDN3 | 219 | 1396 | 215 |
| 4316 | MMP7 | 258 | 1397 | 114 |
| 10103 | TSPAN1 | 231 | 1215 | 238 |
| 54102 | CLIC6 | 41 | 184 | 32 |
| 54716 | SLC6A20 | 52 | 241 | 69 |
| 25805 | BAMBI | 82 | 286 | 67 |
| 3595 | IL12RB2 | 32 | 110 | 26 |
| 346389 | MACC1 | 145 | 489 | 76 |
| 6372 | CXCL6 | 20 | 68 | 21 |
| 2239 | GPC4 | 116 | 378 | 69 |
| 5789 | PTPRD | 71 | 256 | 81 |
| 3489 | IGFBP6 | 77 | 238 | 71 |
| 3918 | LAMC2 | 53 | 182 | 60 |
| 63917 | GALNT11 | 225 | 659 | 209 |
| 135932 | TMEM139 | 99 | 280 | 88 |
| 5069 | PAPPA | 63 | 174 | 55 |
| 30811 | HUNK | 60 | 164 | 43 |
| 6662 | SOX9 | 180 | 492 | 117 |
| 83543 | AIF1L | 105 | 283 | 77 |
| 54825 | CDHR2 | 64 | 169 | 61 |
| 144165 | PRICKLE1 | 72 | 179 | 69 |
| 6659 | SOX4 | 216 | 534 | 174 |
| 27445 | PCLO | 51 | 141 | 57 |
| 54756 | IL17RD | 53 | 130 | 51 |
| 4634 | MYL3 | 65 | 160 | 66 |
| 400566 | C17orf97 | 54 | 133 | 55 |
| 108 | ADCY2 | 31 | 75 | 30 |
| 5745 | PTH1R | 79 | 184 | 71 |
| 80162 | PGGHG | 127 | 293 | 114 |
| 166647 | ADGRA3 | 116 | 266 | 90 |
| 1999 | ELF3 | 175 | 398 | 164 |
| 3934 | LCN2 | 55 | 243 | 109 |
| 81615 | TMEM163 | 54 | 119 | 48 |
| 3912 | LAMB1 | 429 | 1017 | 462 |
| 23359 | FAM189A1 | 56 | 134 | 62 |
| 51435 | SCARA3 | 113 | 241 | 88 |
| 10317 | B3GALT5 | 32 | 74 | 35 |
| 5076 | PAX2 | 173 | 369 | 143 |
| 6565 | SLC15A2 | 41 | 87 | 30 |
| 54768 | HYDIN | 33 | 69 | 29 |
| 94234 | FOXQ1 | 79 | 166 | 76 |
| 122481 | AK7 | 27 | 56 | 22 |
| 5364 | PLXNB1 | 106 | 260 | 126 |
| 131566 | DCBLD2 | 240 | 489 | 171 |
| 56245 | C21orf62 | 52 | 105 | 52 |
| 222236 | NAPEPLD | 82 | 164 | 65 |
| 152330 | CNTN4 | 44 | 88 | 33 |

TABLE 2
ccRCC-specific

| GeneID | Symbol | ccRCC | pRCC | chRCC |
|---|---|---|---|---|
| 1356 | CP | 1572 | 18 | 15 |
| 4015 | LOX | 1128 | 40 | 44 |
| 112399 | EGLN3 | 1629 | 126 | 77 |
| 1573 | CYP2J2 | 255 | 23 | 19 |
| 1038 | CDR1 | 554 | 51 | 37 |
| 56901 | NDUFA4L2 | 1018 | 84 | 101 |
| 51129 | ANGPTL4 | 717 | 72 | 69 |
| 159963 | SLC5A12 | 248 | 26 | 26 |
| 2487 | FRZB | 651 | 69 | 57 |
| 2938 | GSTA1 | 175 | 18 | 19 |
| 8701 | DNAH11 | 168 | 17 | 20 |
| 10050 | SLC17A4 | 248 | 29 | 20 |
| 5350 | PLN | 161 | 18 | 20 |
| 23236 | PLCB1 | 414 | 52 | 44 |
| 29923 | HILPDA | 805 | 101 | 76 |
| 1244 | ABCC2 | 167 | 21 | 19 |
| 29974 | A1CF | 169 | 22 | 16 |
| 5054 | SERPINE1 | 581 | 71 | 76 |
| 6515 | SLC2A3 | 806 | 107 | 85 |
| 115361 | GBP4 | 448 | 48 | 59 |
| 4311 | MME | 221 | 32 | 26 |
| 2157 | F8 | 315 | 43 | 47 |
| 2327 | FMO2 | 246 | 38 | 21 |
| 10203 | CALCRL | 359 | 57 | 56 |
| 54578 | UGT1A6 | 345 | 55 | 22 |
| 8564 | KMO | 103 | 18 | 12 |
| 9457 | FHL5 | 132 | 18 | 23 |
| 7903 | ST8SIA4 | 246 | 43 | 37 |
| 6513 | SLC2A1 | 818 | 154 | 144 |
| 80243 | PREX2 | 184 | 26 | 36 |
| 3486 | IGFBP3 | 3445 | 683 | 635 |
| 230 | ALDOC | 320 | 61 | 64 |
| 5166 | PDK4 | 756 | 156 | 115 |
| 1009 | CDH11 | 266 | 55 | 51 |
| 3625 | INHBB | 247 | 38 | 52 |
| 1906 | EDN1 | 347 | 74 | 50 |
| 55076 | TMEM45A | 110 | 24 | 21 |
| 6531 | SLC6A3 | 334 | 67 | 73 |
| 168537 | GIMAP7 | 366 | 68 | 80 |
| 10964 | IFI44L | 250 | 57 | 29 |
| 57493 | HEG1 | 632 | 133 | 144 |
| 5033 | P4HA1 | 711 | 162 | 147 |
| 55303 | GIMAP4 | 358 | 82 | 73 |
| 2113 | ETS1 | 476 | 111 | 91 |
| 10186 | LHFPL6 | 906 | 170 | 212 |
| 8519 | IFITM1 | 905 | 192 | 222 |
| 57561 | ARRDC3 | 1315 | 326 | 262 |
| 768 | CA9 | 251 | 63 | 62 |
| 6518 | SLC2A5 | 332 | 83 | 55 |
| 664 | BNIP3 | 798 | 201 | 164 |
| 3678 | ITGA5 | 450 | 114 | 107 |
| 23516 | SLC39A14 | 599 | 155 | 133 |
| 11067 | DEPP1 | 655 | 121 | 170 |
| 3910 | LAMA4 | 618 | 93 | 160 |
| 5256 | PHKA2 | 421 | 104 | 110 |
| 6925 | TCF4 | 344 | 81 | 90 |
| 5139 | PDE3A | 117 | 29 | 31 |
| 2 | A2M | 2275 | 549 | 597 |

TABLE 3
chRCC-specific

| GeneID | Symbol | ccRCC | pRCC | chRCC |
|---|---|---|---|---|
| 245972 | ATP6V0D2 | 16 | 18 | 5252 |
| 115111 | SLC26A7 | 12 | 11 | 1241 |
| 5816 | PVALB | 26 | 29 | 2834 |
| 26228 | STAP1 | 12 | 11 | 1107 |
| 1080 | CFTR | 11 | 13 | 805 |
| 253012 | HEPACAM2 | 9 | 10 | 578 |
| 121506 | ERP27 | 78 | 67 | 4154 |
| 116449 | CLNK | 18 | 18 | 943 |
| 51458 | RHCG | 44 | 44 | 1984 |
| 9073 | CLDN8 | 8 | 8 | 228 |
| 127124 | ATP6V1G3 | 6 | 6 | 134 |
| 2299 | FOXI1 | 32 | 37 | 782 |
| 245973 | ATP6V1C2 | 28 | 29 | 532 |
| 158401 | SHOC1 | 9 | 9 | 134 |
| 120939 | TMEM52B | 34 | 38 | 593 |
| 155006 | TMEM213 | 34 | 46 | 698 |
| 658 | BMPR1B | 20 | 18 | 300 |
| 200958 | MUC20 | 158 | 113 | 2314 |
| 6521 | SLC4A1 | 59 | 68 | 995 |
| 7113 | TMPRSS2 | 34 | 42 | 560 |
| 100820829 | MYZAP | 21 | 20 | 273 |
| 525 | ATP6V1B1 | 59 | 68 | 865 |
| 80157 | CWH43 | 20 | 21 | 258 |
| 1950 | EGF | 35 | 25 | 421 |
| 1160 | CKMT2 | 40 | 44 | 464 |
| 2888 | GRB14 | 57 | 57 | 571 |
| 222545 | GPRC6A | 10 | 11 | 111 |
| 10512 | SEMA3C | 94 | 72 | 891 |
| 9194 | SLC16A7 | 68 | 78 | 730 |
| 6549 | SLC9A2 | 13 | 17 | 155 |
| 129684 | CNTNAP5 | 25 | 28 | 254 |
| 3816 | KLK1 | 47 | 53 | 468 |
| 27199 | OXGR1 | 14 | 16 | 144 |
| 23327 | NEDD4L | 201 | 213 | 1651 |
| 5618 | PRLR | 38 | 30 | 293 |
| 266722 | HS6ST3 | 32 | 30 | 240 |
| 154091 | SLC2A12 | 19 | 20 | 150 |
| 51635 | DHRS7 | 203 | 198 | 1485 |
| 199920 | FYB2 | 12 | 11 | 90 |
| 55026 | TMEM255A | 68 | 91 | 635 |
| 10655 | DMRT2 | 29 | 33 | 228 |
| 84803 | GPAT3 | 67 | 74 | 515 |
| 7069 | THRSP | 41 | 45 | 308 |
| 53828 | FXYD4 | 46 | 52 | 352 |
| 202374 | STK32A | 19 | 20 | 135 |
| 202151 | RANBP3L | 13 | 11 | 83 |
| 26049 | FAM169A | 16 | 17 | 112 |
| 51760 | SYT17 | 48 | 66 | 429 |
| 26249 | KLHL3 | 44 | 39 | 277 |
| 57533 | TBC1D14 | 220 | 187 | 1362 |
| 7368 | UGT8 | 93 | 84 | 574 |
| 54899 | PXK | 87 | 97 | 585 |
| 65267 | WNK3 | 21 | 19 | 126 |
| 64409 | GALNT17 | 37 | 43 | 250 |
| 135138 | PACRG | 28 | 36 | 205 |
| 3606 | IL18 | 114 | 118 | 675 |
| 5569 | PKIA | 29 | 31 | 179 |
| 9068 | ANGPTL1 | 14 | 14 | 75 |

### Deconvolution of the TCGA RCC Cohort

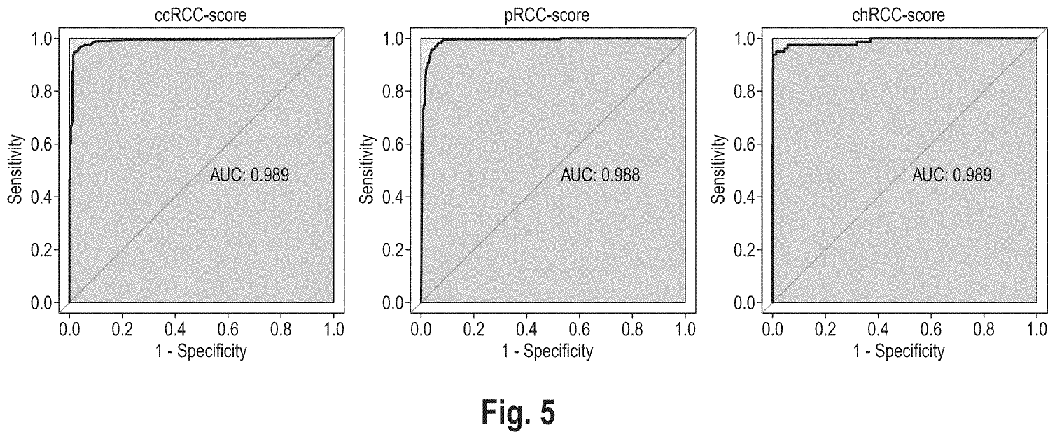

RCC58 was used to perform proportional subtype assignment (PSA) by deconvolution of 864 tumor transcriptomes from the combined TCGA RCC cohort, including the KIRC, KIRP, and KICH cohorts. Receiver operating characteristic (ROC) analyses showed very good agreement between PSA and the most recent histological classification of the TCGA RCC cohort (Ricketts et al., Cell Reports, 2018) (FIG. 5). Note that histological classification can still contain errors.

### Prognostic Classification of RCC Based on PSA

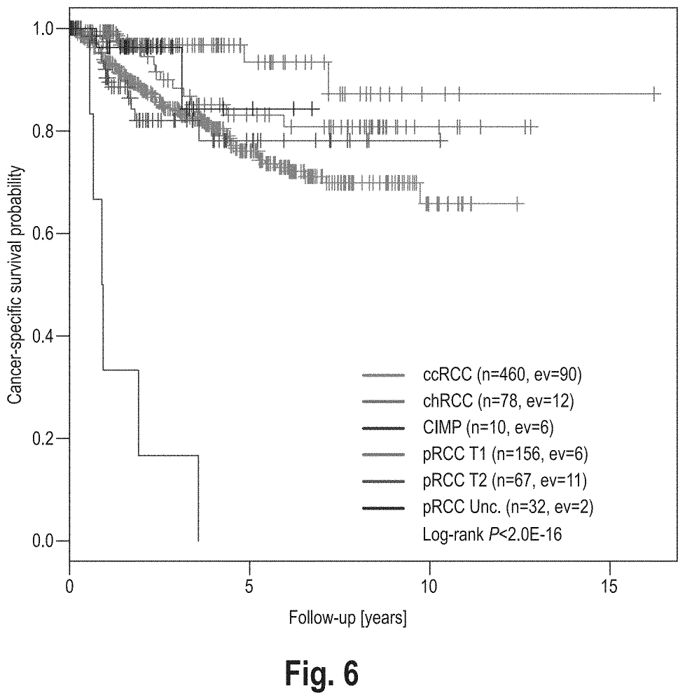

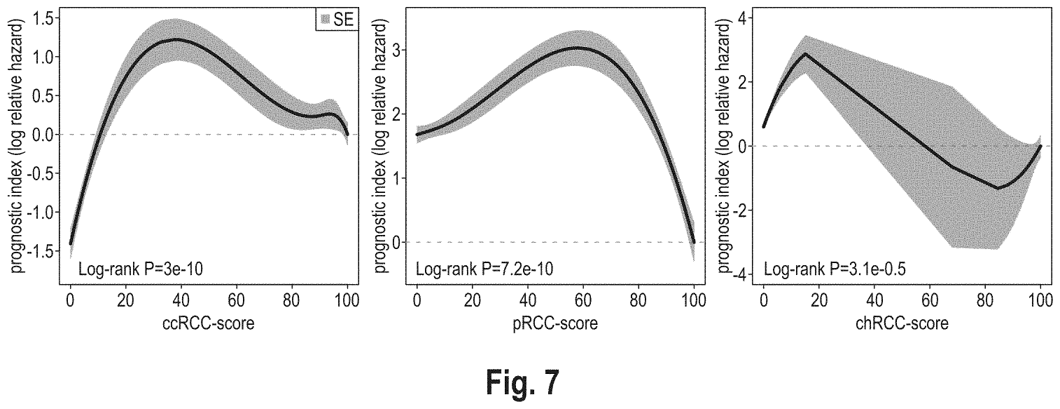

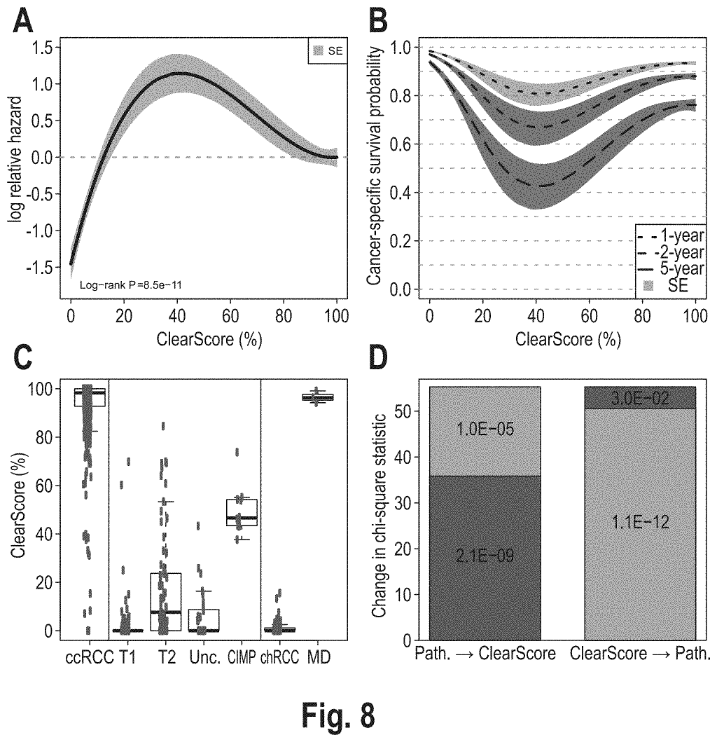

RCC subtypes vary in prognosis (C. J. Ricketts et al., loc. cit.) (FIG. 6). Hence, the inventors were wondering if PSA estimated by deconvolution were predictive of patient survival as well. Deconvolution assigns three estimates (scores) to each sample representing the proportions of ccRCC, pRCC, and chRCC. The terms “proportions” and “scores” are used interchangeably in the following. Univariate Cox proportional hazard regression with subtype scores as continuous predictors were performed in C3. The scores were modelled via restricted cubic spline functions to detect possible non-linear associations. Highly significant, non-linear relationships to CSS were found for the ccRCC-score and the pRCC-score (FIG. 7). The ccRCC-score exhibited the strongest relationship to patient survival and therefore will be presented here in more detail. Analysis of the fitted curve in FIG. 7 suggested a cubic relationship between ccRCC-score and log relative hazard. This observation could be confirmed by the use of a cubic polynomial, which enabled a similarly good fit (FIG. 8A). FIG. 8B shows the estimated 1-, 2-, and 5-year survival rates in dependence of the ccRCC-score (“ClearScore”). Patients with ClearScore between 20 and 70 had worst prognosis. In particular CIMP but also pRCC type 2 and a few of the ccRCC tumors were in this interval (FIG. 8C). Comparing the prognostic value of ClearScore and histological classification revealed that both provide independent information, however, the ClearScore outperformed the pathological classification in cohort C3 (FIG. 8D).

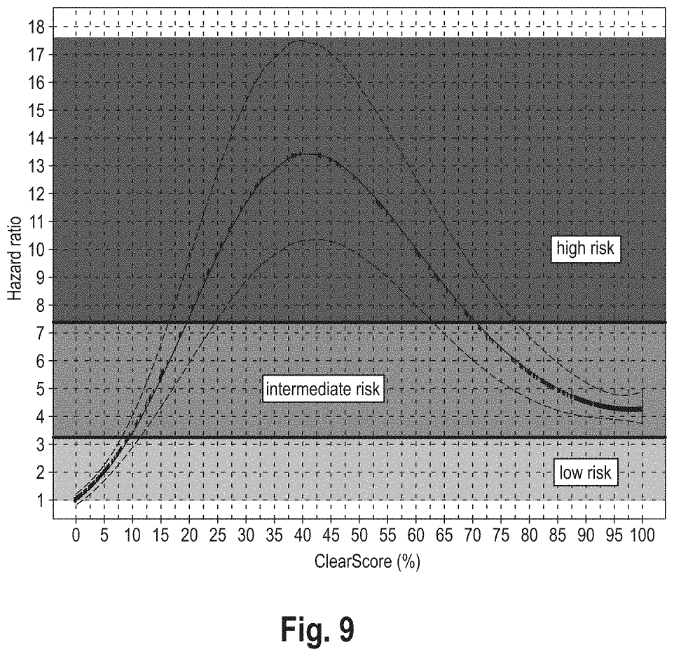

In FIG. 9 the graph illustrates the estimated relationship between ClearScore and the hazard ratio for endpoint cancer-specific death, using a ClearScore of 0% as reference (i.e., a ClearScore of 0%, the hazard ratio was set to 1). The hazard ratio is calculated by taking the exponential of the log relative hazards from FIG. 8A. For example, a hazard ratio of 3 means that the risk of cancer-specific death is three times higher compared to patients having a ClearScore of 0%. The ClearScore allows a classification of the patients into a “high risk” (top area), “low risk” (bottom area) and “intermediate risk” (middle area) group.

### Association Between Survival and Proportional Subtype Assignments Using Different Signature Gene Subsets

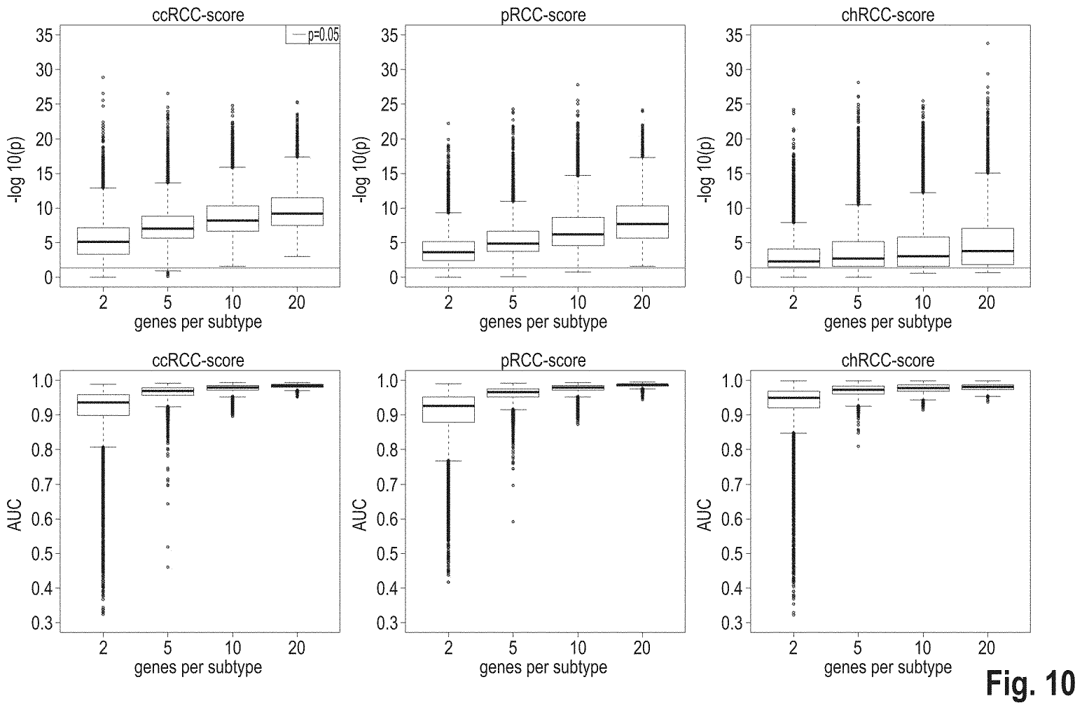

A question that arose was whether subsets of the 3×58 (=174) signature genes are already sufficient for determining in a subject's biological sample the relative proportions of ccRCC, pRCC, and chRCC and whether PSA based on these subsets are significantly associated to survival. The inventors proceeded as follows: The 174 genes are composed of the 58 top-specific genes per subtype. Random subsets of size 3×2 (i.e. 6 genes in total), 3×5, 3×10, and 3×20 were drawn from the set of 3×58 signature genes, i.e. the number of randomly drawn signature genes per subtype were identical with each subset. Random sampling was repeated 10,000 times. With each subset a deconvolution of the TCGA cohort (n=864) was performed, followed by ROC analysis (n=819) and survival time analysis (n=847) as done for the complete signature. Log-rank P-values from survival time analysis and area under the curve (AUC) values are shown in FIG. 10. A clear tendency was observed. With increasing subset size P-values from survival time analysis were decreasing and AUC values were increasing. Even for the majority of the 3×2 gene subsets the association with survival (CSS) was significant as well as the AUC value above 0.9. The inventors assume that non-tested subset sizes would match the trend described here.

Computational gene expression deconvolution is performed by solving a linear system of equations using regression methods such as least square regression, support vector regression, or preferably robust linear regression. In order to derive estimates of the three proportions (pRCC, ccRCC, and chRCC), at least three equations in the linear system are necessary, corresponding to three genes in the signature matrix. In case of three genes in the signature matrix, a sufficient condition for the linear system to have a solution is that these equations (i.e. the rows of the matrix) are linear independent. In our method, this condition can be satisfied by an appropriate selection of three genes, each specific in exactly one of the subtypes.

As a consequence, even with only one gene per subgroup type, i.e. from each of Table 1, Table 2, and Table 3, preferably with two genes per subgroup type a reliable subtype classification can be made.

### Clustering a RCC Cohort by Using Principal Component Analysis (PCA)

The inventors tested whether signal separation methods other than deconvolution can be used to carry out the invention on the basis of the 3×58 (=174) signature genes. Deconvolution makes it possible to analyze individual samples; a forecast is then made on the basis of the relative proportions. Alternatively, the 174 genes can be used to cluster a comprehensive RCC cohort or make a principal component analysis (PCA) or use other techniques from the field of machine learning to group the data. The clustering obtained could then be used as a reference for new, unknown samples: One measures the 174 genes in a new sample, uses their expression levels to cluster the new sample together with the reference cohort, and finally determines the cluster of the new sample. This can be illustrated by a PCA plot. It shows the result of a PCA with the TCGA cohort based on the 174 signature genes according to the invention. The samples are colored according to their relative hazard ratio. One can spot that the samples with similar hazard ratio cluster together.

### Implementation of the Invention into Daily Clinical Routine

Tissue (either fresh, fresh-frozen or FFPE) or body fluids like blood plasma or urine of a patient with RCC is obtained. It may be subjected to the hospital's laboratory or delivered from the hospital or out-patient center to a specialized laboratory. Nucleic acids (total RNA) will be prepared by standard methods. Quantification of the expression levels of candidate genes will be performed using state-of-the art methods. Here different methods like RNA sequencing, microarray or chip based technology or RT-PCR etc. can be used. Based on the established gene signature, (deconvolution-) analysis using well-established algorithms (e.g. robust linear regression) will be performed to determine the proportions of ccRCC, pRCC and chRCC in the sample, subsequently resulting in the outcome classification of RCC patients (low, intermediate, high risk). This report will be delivered to the respective physician, who requested the analyses of RCC specimen.

### Example for Making a Diagnosis by the Method According to the Invention

In the following an example is provided in which on the basis of two genes per RCC subgroup a diagnosis is made for a patient suffering from RCC. The tissue sample of the patient is referred to as TCGA-BQ-5894-01A-11R-1592-07.

Random selection of the two top specific genes per subtype results in the reduced signature matrix RCC2:

| RCC2 | pRCC | ccRCC | chRCC |
|---|---|---|---|
| IL17RD | 130 | 53 | 51 |
| GALNT11 | 659 | 225 | 209 |
| NDUFA4L2 | 84 | 1018 | 101 |
| KMO | 18 | 103 | 12 |
| WNK3 | 19 | 21 | 126 |
| RANBP3L | 11 | 13 | 83 |

For deconvolution, columns of _{RCC2}RCC2 are centered to zero mean and scaled to unit variance, resulting in the scaled signature matrix RCC2_z:

| RCC2_z | pRCC | ccRCC | chRCC |
|---|---|---|---|
| IL17RD | −0.09 | −0.48 | −0.68 |
| GALNT11 | 2.01 | −0.04 | 1.65 |
| NDUFA4L2 | −0.28 | 2.00 | 0.06 |
| KMO | −0.54 | −0.35 | −1.25 |
| WNK3 | −0.53 | −0.56 | 0.43 |
| RANBP3L | −0.57 | −0.58 | −0.21 |

PSA are calculated for sample TCGA-BQ-5894-01A-11R-1592-07 from TCGA KIRC cohort. FPKM-UQ expression values were obtained from https://portal.gdc.cancer.gov/. For the six genes in RCC2_z, the vector m containing these values is given by:

| m | TCGA- BQ-5894-01A-11R-1592-07 |
|---|---|
| IL17RD | 37063.62 |
| GALNT11 | 200584.18 |
| NDUFA4L2 | 230892.48 |
| KMO | 5837.22 |
| WNK3 | 6013.57 |
| RANBP3L | 2038.63 |

As for the signature matrix, values in [symbol] are centered to zero mean and scaled to unit variance, resulting in the scaled expression profile m_z:

| m_z | TCGA-BQ-5894-01A-11R-1592-07 |
|---|---|
| IL17RD | −0.41 |
| GALNT11 | 1.13 |
| NDUFA4L2 | 1.42 |
| KMO | −0.70 |
| WNK3 | −0.70 |
| RANBP3L | −0.74 |

The assumption is that the expression level of gene [symbol] in [symbol] is the sum of its expression in the ccRCC, pRCC and chRCC proportions of sample TCGA-BQ-5894-01A-11R-1592-07. Vector ″ shall denote these unknown proportions of ccRCC, pRCC and chRCC. [symbol] f is estimated by solving the linear system m_z=RCC2_z·f using robust linear regression. The function “rlm” from the R-package MASS performs a robust linear regression and is applied as follows:

$$fit = rlm ( m\_z ∼ RCC2\_z , maxit = 200 )$$

Resulting regression coefficients are accessed via fit$ coefficients:

| intercept | pRCC | ccRCC | chRCC |
|---|---|---|---|
| 0.02 | 0.55 | 0.77 | 0.03 |

The intercept is discarded and proportions are calculated by dividing the three estimates by their sum resulting in the following predicted subtype composition of TCGA-BQ-5894-01A-11R-1592-07:

| pRCC | ccRCC | chRCC |
|---|---|---|
| 0.41% | 0.57% | 0.02 |

Using the coefficients from the Cox model including the ccRCC-score (ClearScore) as well as its square and cube as predictors the prognostic index [symbol] (i.e. log relative hazard) for TCGA-BQ-5894-01A-11R-1592-07 can be calculated, with x=0.57:

$$PI = x \times 14.71 - {x}^{2} \times 25.46 + {x}^{3} \times 12.21 - 1.46$$

Subtraction of 1.46 causes tumors with a ccRCC proportion of 100% to obtain a pi of 0. Hence, the obtained pi of 0.91 denotes the log relative hazard compared to tumors with ccRCC proportion of 100%. e^{pi} (here 2.48) gives the hazard ratio between TCGA-BQ-5894-01A-11R-1592-07 and the group of tumors with ccRCC proportion of 100%. In addition, with the baseline survival function the probability of survival at a certain time point can be calculated. For the given example the predicted cancer-specific 1-year survival probability is 84% (SE: 81%-87%), the 2-years survival probability is 73% (SE: 67%-77%), and the 5-year survival probability 51% (SE: 42%-58%).

### Conclusion and Miscellaneous

The inventors provide for the very first time an objective and reference-free subtype classification or a proportional subtype assignment method for RCC which provides reliable results and is easily applicable in clinical settings.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Therefore, what is claimed, is:

1. A method for determining in a subject's biological sample the relative proportions of papillary renal cell carcinoma (pRCC), clear cell renal cell carcinoma (ccRCC), and chromophobe renal cell carcinoma (chRCC), the method comprising:
(a) Providing a biological sample from a subject suspected of being affected by RCC,
(b) Assaying said biological sample to determine expression level values of
at least one of the signature genes listed in Table 1,
at least one of the signature genes listed in Table 2, and
at least one of the signature genes listed in Table 3,
(c) Subjecting the obtained expression level values to a signal separation method, thereby determining relative proportions of pRCC, ccRCC, and chRCC in said biological sample.

2. The method of claim 1, wherein in step (b) said biological sample is assayed to determine expression level values of
at least two of the signature genes listed in Table 1,
at least two of the signature genes listed in Table 2, and
at least two of the signature genes listed in Table 3.

3. The method of claim 1, wherein the signal separation method is a method selected from the group consisting of: blind signal separation method, deconvolution, and computational deconvolution.

4. The method of claim 1, wherein after step (c) the following step is carried out:
(d) Classifying the subject into a risk group on the basis of the relative proportions of at least one of pRCC, ccRCC, and chRCC in said biological sample.

5. The method of claim 1, wherein after step (c) the following step is carried out:
(d) Classifying the subject into a risk group on the basis of the relative proportions of hccRCC in said biological sample.

6. The method of claim 4, wherein the risk group is selected from “low risk”, “intermediate risk”, and “high risk” according to the prognosis for the subject.

7. The method of claim 6, wherein the “low risk” group is determined by a relative ccRCC proportion in a range which is selected from the group consisting of: about ≥0 to ≤12%, about ≥0 to ≤5%, about ≥0 to 3%, and about 0%.

8. The method of claim 6, wherein the “intermediate risk” group is determined by a relative ccRCC proportion in a range which is selected from the group consisting of: about ≥7.5 to ≤25%, about ≥10 to ≤20%, and about ≥13 to ≤17%.

9. The method of claim 6, wherein the “intermediate risk” group is determined by a relative ccRCC proportion in a range which is selected from the group consisting of: about ≥62.5%, about ≥70%, %, about ≥77.5%, about ≥90%, and about 100%.

10. The method of claim 7, wherein the “high risk” group is determined by a relative ccRCC proportion in a range which is selected from the group consisting of: about ≥16 to ≤77.5%, about ≥20 to ≤70%, about ≥55 to ≤62.5%, and about 40%.

11. The method of claim 1, wherein in step (b) the assaying involves the use of RNA sequencing, PCR-based method, microarray-based method, hybridization-based method, and antibody-based method.

12. An array comprising capture molecules capable of specifically binding to
biomolecules encoding or encoded by at least one of the signature genes listed in Table 1 or segments thereof,
biomolecules encoding or encoded by at least one of the signature genes listed in Table 2 or segments thereof, and
biomolecules encoding or encoded by at least one of the signature genes listed in Table 3.

13. An array comprising capture molecules capable of specifically binding to
biomolecules encoding or encoded by at least two of the signature genes listed in Table 1 or segments thereof,
biomolecules encoding or encoded by at least two of the signature genes listed in Table 2 or segments thereof, and
biomolecules encoding or encoded by at least two of the signature genes listed in Table 3.

14. The array of claim 12, wherein said biomolecules are selected from the group consisting of: nucleic acid molecules, proteins, and peptides.

15. The array of claim 13, wherein said biomolecules are selected from the group consisting of: nucleic acid molecules, proteins, and peptides.

16. The array of claim 12, wherein said capture molecules are selected from the group consisting of: nucleic acid molecules, antibodies and fragments thereof.

17. The array of claim 13, wherein said capture molecules are selected from the group consisting of: nucleic acid molecules, antibodies and fragments thereof.
